(19) United States
(12) Patent Application Publication
(10) Pub. No.: US 2003/0018337 A1
(43) Pub. Date: Jan. 23, 2003

(54) Bone drill and tap combination

(76) Inventor: Reginald J. Davis, Baltimore, MD (US)

Correspondence Address: CONLEY, ROSE & TAYON, A PROFESSIONAL CORPORATION, THE CHASE BUILDING, 700 LAVACA, SUITE 800, AUSTIN, TX, 78701-3102, US

(21) Appl. No.: 09/908,456

(22) Filed: Jul. 17, 2001

(51) Int. Cl. A61B 17/00

(52) U.S. Cl. 606/80

(57) ABSTRACT

A drill and tap tool may be used to form threaded openings in bone. The tool may include a shaft that has a drill section located at an end of the shaft. A tap section may be located adjacent to the drill section. The drill section may include a drill point for initiating an opening in a bone. A drill bit section may abut the boring tip, and a tap section may be adjacent to the drill bit section. The drill bit section may be configured to form an opening in the bone. A diameter of the opening formed by the drill bit section may be substantially the same as the shaft diameter of the screw to be inserted in the threaded opening formed by the drill and tap tool.

### BACKGROUND OF THE INVENTION

1. Field of the Invention

The present invention generally relates to drill bits and taps. An embodiment of the invention relates to a single drill and tap instrument that is capable of forming a threaded opening in a bone.

2. Description of the Related Art

Many bone fixation procedures require the attachment of devices and/or plates to a bone. Devices and plates may be attached to a bone with bone screws. Typically, a bone must be prepared to accept a bone screw. The preparation process may be a two step procedure. First, an opening may be drilled in the bone. Then, a thread flight that corresponds to the threading of a bone screw may be formed in the opening with a separate component tapping tool. An interior wall of a threaded opening should be free of cracks so that the threaded opening will securely hold a bone screw that is screwed into the threaded opening.

To prepare a bone to accept a bone screw, an opening may be drilled into the bone with a drill bit. Drill bits typically include a shaft having a cutting section. An end of the shaft is adapted to couple to a device that imparts rotational motion about a central longitudinal axis of the shaft. The device may be a power drill or a hand drill. An opening may be formed in a bone by rotating the drill bit and moving the cutting section of the drill bit into the bone.

The cutting section of a drill bit may include a start and at least one cutting edge. The start may be a pointed section capable of initiating an opening in a bone. Many different types of drill bits may be used to form an opening in a bone including helical bits and spade bits. A helical bit may have a spiral groove or grooves with a cutting edge formed on at least one peripheral edge of each groove. A spade bit may have two separated flat surfaces with a cutting edge formed along an edge of each flat surface.

A tap may be used to form a thread flight in a previously formed opening. To form a thread flight in an interior wall of an opening, a tap may be inserted into an opening, and the tap may be rotated while being driven into the opening. The rotation of the tap forms a thread flight in a wall of the opening. One type of tap may be considered to be a male screw having cutting edges and flutes. The cutting edges form a female thread in a wall of an opening. The flutes of the tap may define the cutting edges, and the flutes may also provide channels for removal of chips that are produced during formation of a female thread flight.

A single tool may be used to form both an opening and an internal thread. U.S. Pat. No. 4,271,554 issued to Grenell, and incorporated by reference as if fully set forth herein, describes a combination drill and tap tool for formation of a threaded hole in ductile metal by coining or flowing the metal. A thread is formed in the metal by flowing, as opposed to cutting, the metal. The thread flight formed by the tool has a minor crest-to-crest diameter that is slightly less than the diameter of the hole formed by the drill portion of the tool. The diameter of the drill portion of the tool is greater than the minimum diameter of the tap portion of the tool. The drill portion is a spade bit that includes lateral grooving. The lateral grooving allows the drill portion to be removed from the threaded hole without shearing the crests of the formed threading.

U.S. Pat. No. 4,761,844 issued to Turchan, and incorporated by reference as if fully set forth herein, describes a combination drill and tap tool for formation of a threaded hole in a workpiece. A drill bit portion of the tool has a diameter that is substantially equal to a maximum diameter of a tap portion of the tool. To form a threaded hole, the drill bit portion is used to form an initial hole in the workpiece. The tool, or the workpiece, is moved along an orbital path to form a threaded interior wall after the initial hole is drilled to a desired depth in the workpiece. The use of a tool that requires orbital motion to form a thread may not be applicable to forming a thread in a bone of a living patient.

### SUMMARY OF THE INVENTION

A unitary tool may be used to form a threaded opening in a bone of a living patient. A threaded opening may be formed using a combination drill and tap tool. The tool may include a shaft that has a drill bit portion adjacent to a tap portion. In an embodiment, the drill bit portion includes a tip and a cutting edge that forms an opening in the bone when the drill bit portion is rotated. The drill bit portion may be used to form an opening in the bone at a desired location along a length of the bone. A mark or indentation may be formed on or in the bone at or near the desired location prior to the use of the tool. A threaded opening in a bone may be formed by creating an opening in a bone with the drill bit portion and forming a thread flight in a wall of the opening with the tap portion. The drill bit portion of the tool may be a wide variety of drill bits, including, but not limited to a helical bit or a spade bit.

A tap portion of a drill and tap tool may be located adjacent to a drill bit portion. The tap portion may be used to form a thread flight in a bone. The tap portion of the tool may include cutting sections that are separated by flutes. Cutting sections of the tap portion may be configured to cut specific types of threads in a bone. For example, the cutting sections of an embodiment of a tool are generally configured to cut threads in a bone that will generally mate with cancellous threading of a bone screw. The types of threading that may be formed by a given configuration of a tap portion may be, but are not limited to, cancellous threading or cortical threading. Embodiments of tap portions may form threading in bone that has a combination of threading types, such as cancellous threading and cortical threading. Embodiments of tap portions may form threading that mate with bone screws having variable diameter shanks.

A drill bit portion of a drill and tap tool may have a diameter that forms a hole within a bone that is smaller than a minor crest-to-crest diameter of thread formed by a tap section of the tool. Bone may be soft enough to compress, yet the bone may also be hard enough to accept formation of threading. Bone may be soft enough for the drill bit portion to advance at a rate that allows the tap portion to form threading in the bone. The threading may be formed at a rate determined by thread pitch of the tap section and by the rotational speed of the drill and tap tool.

An advantage of the combination drill and tap tool includes that a single or unitary tool can be used to form a threaded opening in a bone. Using a unitary tool to form a threaded opening may save time during a surgical procedure. Also, having a unitary tool that forms an opening and a thread flight in the opening may eliminate the need to purchase or have available several separate tools to form a threaded opening in a bone. Another advantage of the tool is that the tap portion of a specific drill and tap tool may be configured to form a desired threading configuration in the bone that will mate with a particular type of bone screw. Other advantages of drill and tap tool embodiments may include that the drill and tap tools are safe, durable, light weight, simple, efficient, reliable and inexpensive; yet the tools may also be easy to manufacture, maintain and use.

### BRIEF DESCRIPTION OF THE DRAWINGS

Further advantages of the present invention will become apparent to those skilled in the art with the benefit of the following detailed description of the preferred embodiments and upon reference to the accompanying drawings in which:

FIG. 1 shows a perspective view of an embodiment of a combination drill and tap tool;

FIG. 2 shows a cross sectional representation of a threaded opening formed by an embodiment of a drill and tap tool;

FIG. 3 shows a front elevational view of an embodiment of a bone screw;

FIG. 4 shows a partial perspective view of an embodiment of a drill and tap tool having a spade drill bit;

FIG. 5 shows a partial perspective view of an embodiment of a drill and tap tool having a helical drill bit; and

FIG. 6 shows an embodiment of a cross sectional representation of a threaded opening adapted to accept a bone screw that has a variable diameter shank.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

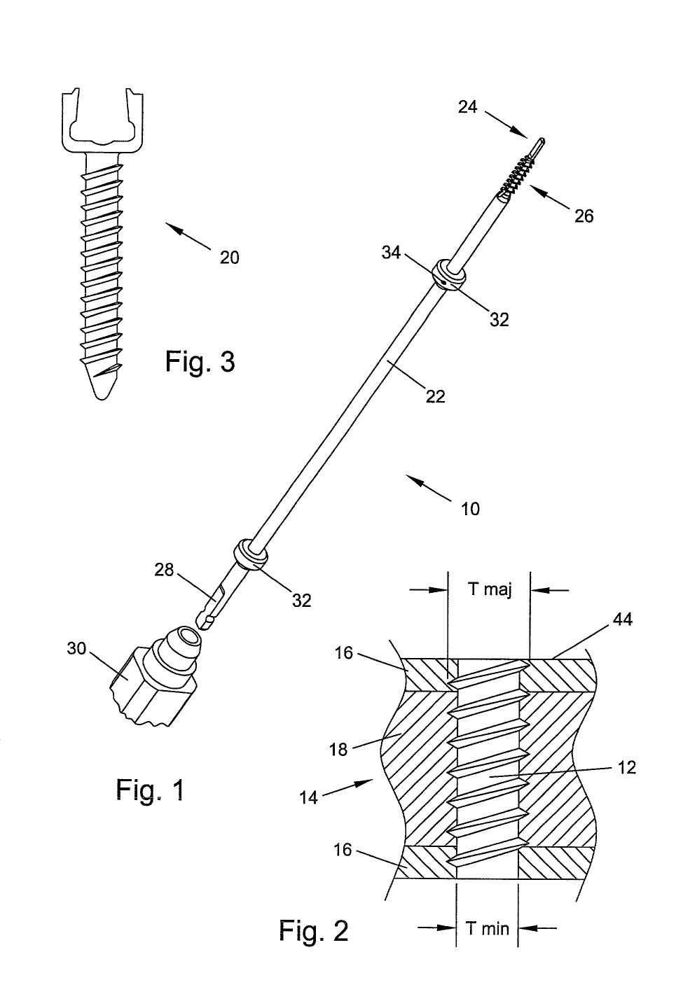

Referring to the drawings, and particularly to FIG. 1, drill and tap tools are designated generally as 10. The tool 10 may be used to form threaded opening 12 in bone 14 in a single operation. A threaded opening 12 that may be formed using an embodiment of the tool 10 is illustrated in FIG. 2. The threaded opening 12 may extend through cortical bone 16 and through cancellous bone 18. A thread flight formed by the tool 10 may be adapted to mate with a threaded fastener, such as a bone screw 20. An embodiment of a bone screw 20 is shown in FIG. 3. Thread formed in a bone 14 by the tool 10 may be, but is not limited to, cancellous threading or cortical threading. Cancellous thread may have a large thread pitch and a large thread depth. Thread depth may be defined as one half the difference between a major crest-to-crest diameter and a minor crest-to-crest diameter of the thread. Cortical thread may have a finer thread pitch and a smaller thread depth. The coarse pitch and large thread depth of cancellous thread may provide good purchase between the bone screw 20 and bone 14 in cancellous bone 18, while the finer pitch and smaller thread depth of cortical thread may provide good purchase between the bone screw and bone in dense bone material.

A drill and tap tool 10 may include shaft 22, drill bit section 24, and tap section 26. End 28 of the shaft 22 may be attached to drill 30. The drill and tap tool 10 may be formed of medical grade metal. The metal may have a minimum Rockwell hardness of about 47 so that cutting surfaces of the tool retain a sharp edge. One type of material that may be used to form the drill and tap tool 10 is heat-treated type 455 stainless steel.

The shaft 22 may optionally include stops 32. A stop 32 may limit an insertion depth of the shaft 22 into the drill 30. A stop 32 may also limit an insertion depth of the drill and tap tool 10 into a bone 14. The stops 32 may be adjustably positionable along a length of the shaft 22, or the stops may be permanently fixed at desired locations on the shaft. In an embodiment of a tool 10, the position of an adjustable stop 32 may be set at a desired location by tightening a setscrew 34 in the body of the stop to press the setscrew against the shaft 22. In an embodiment of a tool 10, a stop 32 may inhibit further drilling and tapping of a bone after the tool has threaded a cortical portion of the bone.

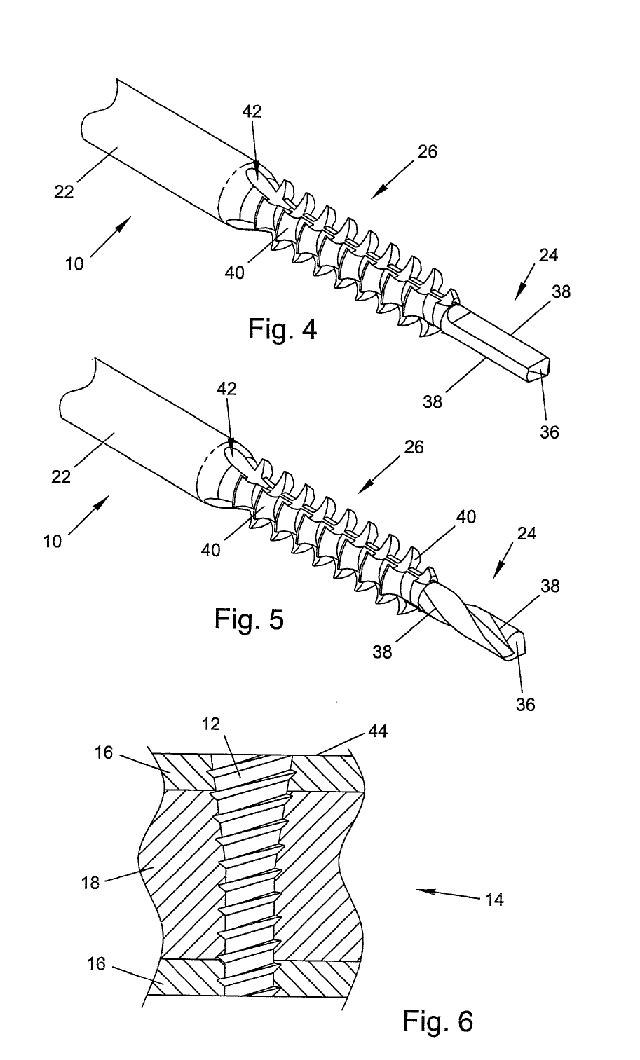

The drill bit section 24 of the drill and tap tool 10 may include tip 36 and cutting edges 38. The drill bit section 24 may be a wide variety of drill bits that can form an opening in bone 14. For example, FIG. 4 shows an embodiment of a portion of a drill and tap tool 10 wherein the drill bit section 24 is a spade drill bit, and FIG. 5 shows an embodiment of a portion of a drill and tap tool 10 wherein the drill bit section 24 is a helical drill bit. The tip 36 may initiate formation of an opening in a bone 14 when a rotating tool 10 is positioned against the bone and pressed into the bone. After the initial opening is formed, cutting edges 38 of the drill bit section 24 may form an opening of a desired diameter in the bone 14 as the drill and tap tool 10 is further inserted into the bone.

An embodiment of a tool 10 may include a drill bit section 24 that is located near an end of shaft 22. A tap section 26 may be located adjacent to the drill bit section 24. In an embodiment, the tap section 26 abuts the drill bit section 24. In an alternate embodiment, a section of shaft 22 with a diameter that is smaller than a diameter cut by the cutting edges 38 of the drill bit section 24 may separate the tap section 26 and the drill bit section. The tool 10 may be configured to tap cortical bone 16 but not cancellous bone 18. The bone screw 20 that is inserted into the opening 12 formed by the embodiment of the tool 10 may have self-tapping cancellous thread on a lower section of a shank and cortical thread on an upper section of the shank.

An embodiment of a tool 10 may have a tap section 26 that is located near an end of the tool. The drill bit section 24 is located above the tap section 26. Such a tool may be pulled into and through a hole or opening to enlarge and thread the hole or opening.

A drill bit section 24 of a drill and tap tool 10 may have a diameter that forms a hole within a bone that is smaller than a minor crest-to-crest diameter of thread formed by a tap section 26 of the tool. Bone may be soft enough for the drill bit section 24 to advance at a rate that allows the tap section 26 to form threading in the bone. The threading may be formed at a rate determined by thread pitch of the tap section 26 and by the rotational speed of the drill and tap tool 10. If the drill bit section advances too slowly into the bone, rotation of the tap section within the bone may produce a larger opening instead of a threaded opening.

Bone may be soft enough to compress, yet the bone may also be hard enough to accept formation of threading. A diameter cut by cutting edges 38 of a tool 10 may be substantially the same as a minor crest-to-crest diameter of a thread flight formed in an opening 12 by the tap section 26. In alternate embodiments, the diameter cut by the cutting edges 38 may be smaller than the minor crest-to-crest diameter of the thread formed by the tap section 26. In embodiments, the diameter cut by the cutting edges 38 is greater than about 70% of the minor crest-to-crest diameter of the thread formed by the tap section 26, or greater than about 80% of the minor crest-to-crest diameter of the thread formed by the tap section, or greater than about 90% of the minor crest-to-crest diameter of the thread formed by the tap section, or greater than about 95-97% of the minor crest-to-crest diameter of the thread formed by the tap section. The tap section 26 may enlarge the diameter cut by the cutting edges 38 of the drill bit section 24. Allowing a portion of cut away bone to be removed and/or compressed by the tap section 26 instead of cutting edges 38 of the drill bit section 24 may help to evenly distribute heat throughout the tool 10 during the formation of the threaded opening 12. A minor crest-to-crest diameter of a thread flight is designated as “T min” in FIG. 2. The minor crest-to-crest diameter of the threaded opening 12 formed by the tool 10 may be substantially the same or smaller than a shank diameter of a bone screw 20 that will be inserted into the threaded opening.

The tap section 26 of the drill and tap tool 10 may include cutting sections 40 that are separated by flutes 42. The tap section 26 may be considered to be a male thread flight that has a plurality of cutting sections 40 separated and defined by flutes 42. The cutting sections 40 form a female thread flight in a bone 14 with major crest-to-crest diameters. A major crest-to-crest diameter of a thread flight is designated as “T maj” in FIG. 2. The flutes 42 may provide a channel for the removal of chips cut during formation of an opening 12 and thread flight in a bone 14. In an embodiment, the tap section has three flutes that are equally spaced around the shaft 22. Fewer, or more, than three flutes may be formed in a shaft 22 of a tool 10 in specific drill and tap tool embodiments. Also, the flutes may be helically formed about a shaft 22 of the tool.

The tap section 26 may be used to form a female thread in a bone 14. In an embodiment of a drill and tap tool 10, a start of the tap section 26 may have an initial diameter that is substantially the same as a maximum diameter of the cutting edges of the drill bit section 24. The diameter of the cutting sections 40 may increase to a diameter that is substantially the same as a major crest-to-crest diameter of thread of a bone screw 20 to be inserted into the threaded opening 12.

Embodiments of drill and tap tools 10 may include cutting sections 40 configured to cut cancellous and/or cortical thread in a bone 14. In an embodiment of a tool that cuts both cancellous and cortical thread, the major crest-to-crest diameter of the cancellous thread may be smaller than the major crest-to-crest diameter of the cortical thread. A stop 32 may limit an insertion depth of the tool 10 into a bone 14 to ensure that cortical and cancellous thread are located in desired sections of the bone.

End 28 of the shaft 22 may be attached to a drill 30. The drill 30 may be a hand operated drill or a power drill. The drill 30 may have a slip clutch that inhibits application of excessive torque to the shaft 22 when the drill and tap tool 10 is forming a threaded opening 12 in a bone 14. A drill and tap tool 10 may advance into a bone at a rate that is related to the revolution speed of the tool and to the thread pitch of threading formed by a tap section 26 of the tool. A hand operated drill or chuck may allow precise control of the revolution speed of the tool 10 to inhibit rapid advancement of the tool into bone. A rotation speed of a chuck of a power drill used with the tool 10 may be adjustable so that the tool cannot rotate faster than a desired rate. The desired rate may be at, or lower than, a maximum advancement rate that allows formation of a thread in the bone with the tap section 26.

A threaded opening 12 produced by a tool 10 may accept a threaded fastener having a variable diameter shank, variable pitch threading and/or variable thread depth. FIG. 6 shows a representation of an opening 12 in a bone 14 formed with a tool 10. Some embodiments of threaded openings 12 produced by a tool 10 are adapted to mate to bone screws 20 having variable diameter shanks, variable pitch threading and/or variable thread depth. For example, the threaded opening 12 in bone 14 depicted in FIG. 6 may be formed using a drill and tap tool 10 that has a tapered tap section 26. A stop 32 may limit an insertion depth of the tool 10 into the bone 14 so that the tapering portion of the tap section 26 only cuts tapering thread near a top of the bone. In other embodiments, a threaded opening 12 formed in a bone by a tool 10 may be further processed with drills, taps and/or other tools to produce a threaded opening capable of mating to various types of threaded fasteners having variable diameter shanks, variable pitch threading, and/or variable depth threading.

To use a drill and tap tool 10 to form a threaded opening 12 in a bone 14, the drill and tap tool may be attached to a drill 30. The drill 30 may be a hand operated drill or a power drill. The drill 30 may be used to rotate the drill and tap tool 10. The tip 36 of the drill and tap tool 10 may be positioned on top surface 44 of a bone 14 (shown in FIG. 2) where a threaded opening 12 is to be formed. The drill and tap tool 10 may be rotated and pressed into the bone 14 so that the drill bit section 24 of the drill and tap tool forms an opening in the bone. The tap section 26 may enter the opening formed by the drill bit section 24. The tap section 26 of the drill and tap tool 10 forms a thread in a wall of the opening as the drill and tap tool is rotated and driven further into the bone 14. After the threaded opening 12 is formed in the bone 14, the drill and tap tool 10 may be backed out of the threaded opening by reversing the direction of rotation imparted to the drill and tap tool by the drill 30.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A tool for forming a threaded opening in a human bone, comprising:
a shaft;
a drill bit located on the shaft, the drill bit configured to form an unthreaded opening in the bone; and
a tap adjacent to the drill bit, the tap configured to form threading in the unthreaded opening;
wherein a diameter of the unthreaded opening in the bone formed by the drill bit is between about 70 to 100 percent of a minor crest-to-crest diameter of thread formed by the tap.

2. The tool of claim 1, wherein the drill bit comprises a tip, and wherein the tip is configured to initiate a hole in a bone.

3. The tool of claim 1, wherein the drill bit comprises a spade drill bit.

4. The tool of claim 1, wherein the drill bit comprises a helical drill bit.

5. The tool of claim 1, wherein an end of the shaft is configured to couple to a drill, wherein the drill bit is located near an opposite end of the shaft, and wherein the tap is located between the end and the drill bit section.

6. The tool of claim 1, wherein an end of the shaft is configured to couple to a drill, wherein the tap is located near an opposite end of the shaft, and wherein the tap is located between the end and the tap section.

7. The tool of claim 1, wherein a section of shaft separates the drill bit from the tap.

8. The tool of claim 1, wherein the drill bit abuts the tap.

9. The tool of claim 1, further comprising a stop positioned on the shaft, the stop configured to limit an insertion distance of the shaft into the bone.

10. The tool of claim 9, wherein the stop is adjustably positionable along a length of the shaft.

11. The tool of claim 1, wherein the tap comprises a plurality of thread cutting sections separated by flutes.

12. The tool of claim 1, wherein the thread flight produced by the tap is adapted to engage thread of a bone screw.

13. The tool of claim 1, wherein the tap is adapted to produce cancellous bone threading.

14. The tool of claim 1, wherein the tap is adapted to produce cortical bone threading.

15. The tool of claim 1, wherein the tap is adapted to produce cancellous bone threading and cortical bone threading.

16. The tool of claim 1, further comprising a drill configured to couple to the shaft.

17. The tool of claim 1, wherein the diameter of the opening in the bone formed by the drill bit is greater than about 80 percent of the minor crest-to-crest diameter of thread formed by the tap.

18. The tool of claim 1, wherein the diameter of the opening in the bone formed by the drill bit is greater than about 90 percent of the minor crest-to-crest diameter of thread formed by the tap.

19. The tool of claim 1, wherein the diameter of the opening in the bone formed by the drill bit is greater than about 95 percent of the minor crest-to-crest diameter of thread formed by the tap.

20. The tool of claim 1, wherein the diameter of the opening in the bone formed by the drill bit is substantially the same as the minor crest-to-crest diameter of thread formed by the tap.

21. A tool for forming a threaded opening in a human bone, comprising:
a tap section on a shaft, the tap section configured to form thread in the bone; and
a drill section on the shaft adjacent to the tap section, the drill section configured to form an initial opening in the bone for the tap section;
wherein the drill section is configured to advance into the bone at a rate that allows the tap section to form threading in the bone with a desired thread pitch.

22. The tool of claim 21, further comprising a stop positioned on the shaft, the stop configured to limit an insertion distance of the shaft into the bone.

23. The tool of claim 22, wherein the stop is adjustably positionable along a portion of a length of the shaft.

24. The tool of claim 21, wherein the drill section is configured to form an opening in the bone, and wherein a diameter of the opening is greater than 80 percent of a minor crest-to-crest diameter of thread formed by the tap section.

25. The tool of claim 21, wherein the drill section is configured to form an opening in the bone, and wherein a diameter of the opening is greater than 90 percent of a minor crest-to-crest diameter of thread formed by the tap section.

26. The tool of claim 21, wherein the drill section is configured to form an opening in the bone, and wherein a diameter of the opening is greater than 95 percent of a minor crest-to-crest diameter of thread formed by the tap section.

27. The tool of claim 21, wherein the drill section is configured to form an opening in the bone, and wherein a diameter of the opening is substantially the same as a minor crest-to-crest diameter of thread formed by the tap section.

28. A method of forming a threaded opening in a bone, comprising:
forming an opening in the bone with a drill bit section of a tool;
moving the tool axially into the bone so that a tap section of the tool engages the opening formed by the drill bit section of the tool; and
forming a thread in a wall of the opening with the tap section of the tool by moving the tool into the opening.

29. The method of claim 28 further comprising forming cortical thread in cortical bone of the bone, and forming an unthreaded opening in cancellous bone of the bone.

30. The method of claim 29 further comprising inserting a bone screw comprising cortical thread and self-tapping cancellous thread into the bone.

31. The method of claim 28 further comprising contacting a stop of the tool against the bone to limit an insertion depth of the tool into the bone.
